(19) United States
(12) Patent Application Publication
(10) Pub. No.: US 2010/0106140 A1
(43) Pub. Date: Apr. 29, 2010

(54) ASSAY CATHETER WITH PRESSURE MONITORING

(75) Inventors: Rick M. Odland, Roseville, MN (US); Scott R. Wilson, Maple Grove, MN (US); Bradford G. Staehle, Minnetonka, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: TWIN STAR MEDICAL, INC., St. Paul, MN (US)

(21) Appl. No.: 12/524,445

(22) PCT Filed: Jan. 25, 2008
(86) PCT No.: PCT/US2008/052070

§ 371 Date: Jul. 24, 2009

Related U.S. Application Data
(60) Provisional application No. 60/886,820, filed on Jan. 26, 2007.

(51) Int. Cl. A61M 25/00; A61B 5/03; A61B 18/04; A61B 18/12; A61N 7/00

(52) U.S. Cl. 604/523; 600/561; 606/27; 606/41; 601/2

(57) ABSTRACT

An apparatus adapted to position a functional device tip, such as a sensor, in a body site having both fluid and non-fluid tissue components, in a manner that substantially mitigates the impingement of non-fluid tissue impingement on the device tip. One such apparatus can be used to both remove fluid from a tissue site, such as a site exhibiting tissue swelling, and to determine tissue pressure, using a single catheter. An apparatus can provide a pressure sensor that is adapted to be positioned and used within the tissue site itself, without substantial impingement by non-fluid tissue, and optionally also provides the ability to concurrently remove and/or deliver fluids or components thereof to or from the tissue site.

### TECHNICAL FIELD

The present invention relates to the use of catheters for assaying and/or providing fluids within the body. In another aspect, the invention relates to methods and apparatuses for monitoring parameters such as tissue pressure within the body by means of functional tips positioned in the tissue itself.

### BACKGROUND OF THE INVENTION

In the treatment of several injuries, such as fractures and head injury, it is advantageous to provide pressure monitoring of various tissue parameters, including for instance, tissue pressure. High tissue pressures can be indicative of poor tissue perfusion, which can lead to tissue necrosis. It is also known that the analysis of interstitial fluids can provide diagnostic information, such as tissue micro dialysis, which is a standard of care for traumatic brain injury at some institutions. The removal of fluid can provide therapeutic value in its own right, as described in Applicant's own prior applications and patents.

Typically, the ability to remove and/or analyze fluids, as compared to determining tissue pressure, is performed by separate or different means and/or apparatuses. For instance, in patients at risk for compartment syndrome, typically extremity fractures, the tissue pressure can be measured by sensors remote from the compartment, using fluid coupled methods. These fluid coupled systems are not user friendly and not well suited for the clinical environment. One method to solve these user problems is to place the sensor at the catheter tip as has been done by others. However, it is important that the sensor not directly engage tissue so as to impinge the sensor with a load in addition to the desired hydrostatic pressure. Impingement of the tissue on a sensor surface can often provide false or incorrect readings. One such approach has been to provide the sensor within a catheter having a recessed side port that can displace tissue from direct sensor contact. This approach has its own drawbacks, however, and does not work for “front looking” fiber optic and other such sensors that do not allow for side port orientations.

Yet other lumen catheters having pressure sensors exist and are typically used in blood or hollow organs, i.e., where the risk of direct tissue impingement is not a particular concern.

In a related aspect, catheters exist that employ ultra filtration as a means of recovering interstitial fluid for analysis. Generally, a hollow fiber membrane material is placed into the tissue and negative pressure is applied so as to transport the fluid through and/or into the lumen of the hollow fiber and then down the length of manifold tube until it reaches the fluid collection reservoir. Generally, the fluid sample to be analyzed is then removed from the fluid collection reservoir. Such methods typically do not allow for analyzing the most clinically relevant interstitial fluid, which is found in the manifold tube or other tubing close to and proximal to the manifold tube, since this clinically relevant fluid is the most recently removed fluid from the tissue. See Applicant's own previous patents and applications, including for instance U.S. Ser. No. 10/508,610 (Publication No. 2005-0165342), which describes, inter alia, the manner in which suitable monitors can be used in an integrated fashion with catheters having semipermeable membranes.

### BRIEF DESCRIPTION OF THE DRAWING

FIG. 1 shows a cross-sectional view of the preferred embodiment of this invention, together with end views (1a) and 1b) taken at two points along the length thereof.

FIG. 2 shows an alternative embodiment in which a plurality of aperatures are provided.

FIG. 3 shows both a preferred embodiment of one apparatus, including connectors, and a blow-up view of its distal tip.

FIG. 4 shows a tear-away sheath to provide access to facilitate the placement of an apparatus of this invention.

FIG. 5 shows an alternative view of an apparatus along the lines of that shown in FIG. 3.

FIG. 6 shows a fluid collection catheter, absent a functional (e.g., sensor) tip, and for use in combination with an apparatus of this invention.

FIG. 7 shows a single housing that incorporates a vacuum source, a pressure monitor, a blood pressure monitor, and a user interface.

FIG. 8 shows a clinical set-up of a preferred apparatus and pressure monitoring module.

FIG. 9 illustrates set up configurations for intra-compartment pressuring monitoring using a preferred apparatus and monitoring system

### SUMMARY OF THE INVENTION

In one embodiment, the present invention provides an apparatus that permits a functional device tip, e.g., sensor surface, to be positioned and used within a body, and in contact with a tissue site that comprises both fluid and non-fluid tissue, in a manner that permits fluid tissue to contact the sensor surface but that substantially minimizes direct impingement of the non-fluid tissue on the functional device tip surface. The terms “fluid” and “non-fluid” tissues, as used in this context, will generally refer, respectively, to the difference between tissue that is intended and able to be take up by, or into, an apparatus of this invention, in order to be measured, removed, etc., at or by the functional device tip, as compared to tissue(s) that instead have the tendency or risk of impinging upon or occluding such an apparatus, to the point where a functional device tip can not be used for its intended purpose.

In a preferred embodiment, the apparatus comprises one or more sensors within a catheter, for use in determining one or more corresponding parameters such as tissue pressure, the catheter being configured and used in a manner that permits the apparatus to be positioned in tissue in a manner that permits the sensor(s) to effectively contact fluid tissue within the site, but that minimizes the extent to which the sensor(s) can be impinged upon by non-fluid tissue itself.

In a particularly preferred embodiment, the apparatus also includes one or more conduits, e.g., solid conduits or semipermeable membranes, which provide the ability to either deliver and/or remove fluids and/or components thereof, to or from the tissue site, as for therapeutic and/or analytical purposes. In one such preferred embodiment, the invention provides one or more sensors (e.g., a pressure sensor) adapted to be positioned and used within the tissue site itself, without substantial impingement or occlusion by non-fluid tissue that may be present. Preferably, the catheter also provides the ability to deliver materials (e.g., active agents) and/or remove fluid from a point within the tissue site (e.g., proximal or distal to the sensor surface), thereby providing improved clinical relevance as compared to conventional apparatuses in which both functions, and corresponding structures, are not integrated in a single apparatus.

Alternatively, the apparatus itself is considered novel, and can be used, solely for the purpose of improved sensor placement and use. More preferably, the catheter of this invention provides both improved sensor placement and use, as well as fluid delivery/removal means, and in turn, corresponding options adapted to improve clinical relevance.

### DETAILED DESCRIPTION

An apparatus of this invention permits the use of a sensor needing to have direct contact with tissue fluids to be placed within or amidst non-fluid tissue itself, in a manner that substantially prevents non-fluid tissue from impinging upon the sensor surface. In one preferred embodiment, the sensor tip is placed sufficiently back (proximal) from an exposed tip of the apparatus, in order to let fluid, but not non-fluid tissue, access the sensor itself. In turn, in order to avoid the creation of an air bubble or other artifact within the exposed apparatus tip, one or more suitable vents are preferably positioned between the sensor itself and the apparatus tip, thereby permitting what little air or artifact there may be in the apparatus tip to escape as the tip itself is filled with fluid.

The apparatus comprises one or more sensors that can be positioned within the catheter, and in turn, within the tissue site, in a manner that permits its use without occluding necessary openings or pores, or direct tissue impingement. In one preferred embodiment, the catheter permits a pressure sensor to itself remain separated from direct tissue contact, yet in sufficient fluid communication with the relevant tissue site, in order to permit pressure to be measured accurately, yet not in direct contact with the tissue site itself. For instance, in one preferred embodiment the pressure sensor is a fiber optic sensor incorporating white-light polarization interferometry technology, and is encased within the apparatus tip within a protective material adapted to protect the sensor tip, yet permit pressure to be accurately transferred to it from surrounding fluid.

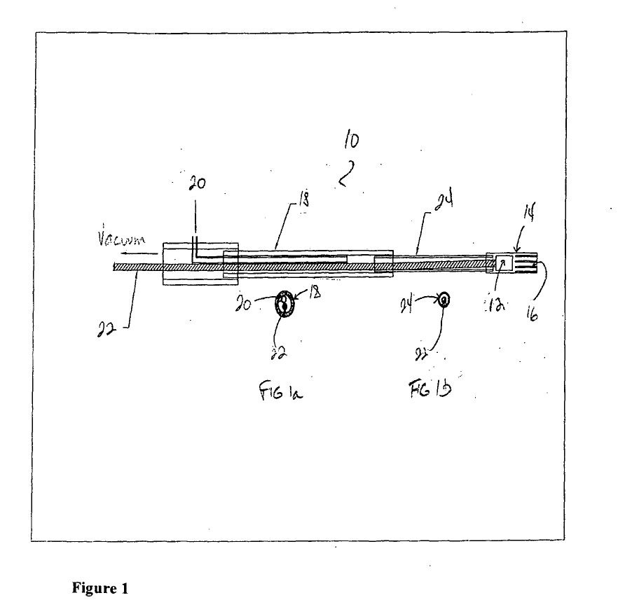

As seen in a preferred embodiment of FIG. 1, for instance, the sensor, including the surface thereof, can be encased in a suitable substance (e.g., silicone gel). The substance provides sufficient physical parameters (e.g., stability, stiffness) to permit pressure to be accurately transferred to, and hence sensed by, the sensor surface.

Sensors for use in a catheter of this invention can be of any suitable type and configuration, e.g., for use in monitoring pressure, pH, temperature, oxygenation, potassium or other electrolytes, biomarkers, optical spectroscopy parameters, tissue impedance, and so on. Optionally, or additionally, the sensor can have or provide a functional aspect as well, e.g., by providing heat, ultrasound and/or an electrical signal sufficient to treat the corresponding tissue site or surface (e.g., by breaking up clots, electroporation, and the like).

Suitable sensors can be based on any technology, e.g., fiber optic, electronic chips, ultrasound, and are preferably fiber optic based sensors adapted to by means of ‘white light interferometry’. See, for instance, “Miniature Fiber Optic Pressure Sensor for Medical Applications: an Opportunity for Intra-Aortic Balloon Pumping (IABP) Therapy”, E. Pinet et al. and “Opsens White-light Polarization Interferometry Technology”, Opsens, Inc., the entire disclosures of both of which are incorporated herein by reference.

Suitable fiber optic sensors are commercially available, e.g., as the “FOP-MIV” sensor available from Fiso Technologies, Inc., which is described as a front looking sensor that allows in situ measurements at locations unreachable to standard pressure sensors. Suitable fiber optic sensors provide an optimal combination of such features as durability and reliability, low cost, ease of use, miniature size, mounting flexibility, resolution, consistency, accuracy and precision, reading rate, fast response, low drift value, and the ability to provide a clear definition of complex pressure waveforms, as well as immunity to electromagnetic field or radiofrequency interference.

The apparatus preferably further comprises one or more lumen for delivering and/or removing fluid from the tissue site, preferably by means of a hollow fiber, and more preferably further comprises one or more vent or shunting means located distally, in order to permit sampling and/or removal of fluids having optimal clinical relevance.

In a particularly preferred embodiment, the apparatus includes a functional tip provided by a catheter that further comprises one or more conduits which provide the ability to either deliver and/or remove fluids and/or components thereof, for therapeutic and/or analytical purposes. In turn, it is quite preferable that the timing and position of the delivery and/or removal of fluids (including components) to or from the tissue site is integrated with the location and function of the functional tip, e.g., such that fluids or active agents intended to alleviate tissue swelling are removed from and/or delivered to the tissue site, in a manner that corresponds with readings generated by the pressure sensor itself.

A preferred embodiment will be described with respect to FIG. 1, in which an apparatus (10) of the present invention includes the use of a suitable sensor (12) associated with and positioned at the distal end of an optical fiber (22) and within a distal portion (14) having slits (16) or other suitable means for permitting fluid communication between the sensor and tissue surrounding the distal portion. As shown, the sensor is displaced back from the most proximal portion of the slits. The tubular distal portion serves to protect the sensor from direct tissue impingement. The slit (16) prevent occlusion of the tube and maintains communication contact with the tissue pressure, due to the bending of the slits when impinging tissue, allowing the sensor fluid contact through the slits.

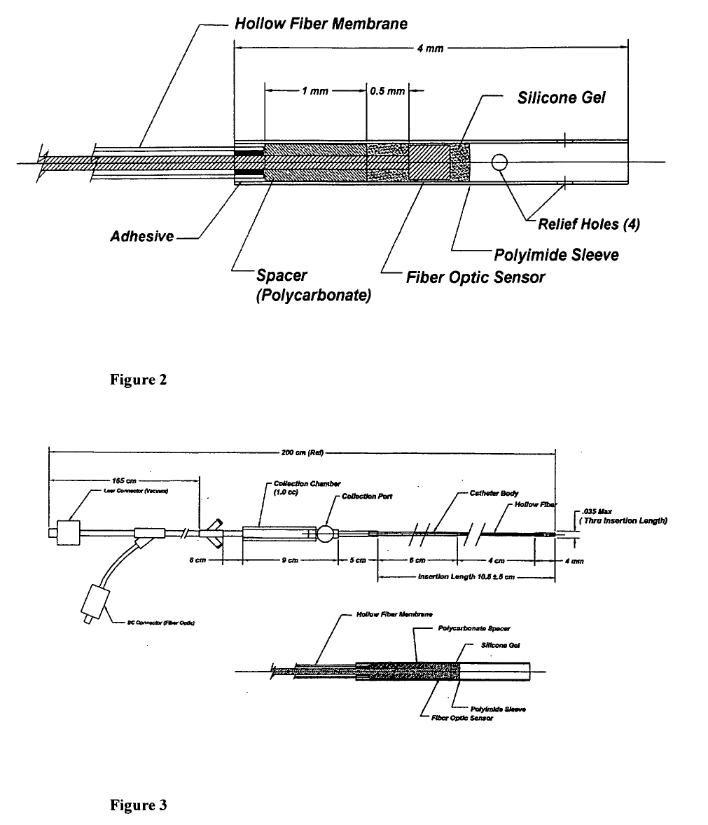

An alternative preferred embodiment is shown in FIG. 2, in which the distal portion of the apparatus is provided with a plurality of apertures for permitting fluid communication between the sensor and tissue surrounding the distal portion. As with the slits of FIG. 1, the apertures prevent occlusion of the tube and maintain communication contact with the tissue pressure, allowing air to vent from the tip, and in turn, permitting the sensor fluid contact throughout the apparatus tip. FIG. 3, in turn, shows an overall apparatus, including connections, as well as enlarged view of the distal end, though absent any particular venting means.

An apparatus of this invention can be prepared using any suitable techniques, e.g., the various parts can be provided separately and assembled in a suitable manner. Alternatively, various combinations and subcombinations of parts can be provided as integral parts, to be finally assembled with others.

The sensors, e.g., fiber optic sensors, and other components for use in the apparatus of this invention can include miniature, micro- and even nanotechnology components for use in minimally invasive diagnosis, therapy, and monitoring, including for instance, physical sensors that are linked to a telemetric unit for wireless data transmission. Such sensors can be biocompatibly packaged or implanted and used in a minimally invasive procedure, to determine such parameters as pressure and/or constituent levels in the blood or tissue itself, temperature, and/or tissue (e.g., nerve) function, and other suitable biological parameters.

An apparatus of this invention can be used, for instance, for the removal of interstitial fluid in order to lower muscle compartment pressure and thereby possibly reducing the need for surgical fasciotomy. For instance, patients that have suffered an isolated tibial fracture (open or closed) typically require surgical stabilization within 72 hours of injury. Such patients can receive an apparatus of this invention, which can be inserted at the end of the surgical procedure to stabilize the tibia fracture and can be connected to the pressure monitor before leaving the surgical room.

The apparatus can be used for the first hours or days following surgical fixation of the affected lower leg in order to: (1) measure and record muscle compartment pressure, and (2) remove accumulated interstitial fluid. Such patients can be treated in any suitable manner, for instance, receiving constant or intermittent vacuum, at the same or varying levels, and optionally, in combination with fluid removal. The apparatus can be provided as either a single-fiber catheters or as multi-fiber catheters.

Situations in which the fluid removal capabilities are employed will typically result in a greater reduction in muscle compartment pressure, as compared to monitoring alone. Samples of the interstitial fluid removed from the patient's leg can be analyzed for various indicators of muscle injury, as well as to determine the serum levels of the same targeted analytes. Interstitial fluid and blood serum levels of the analytes can be correlated to intramuscular pressure levels and other parameters as well.

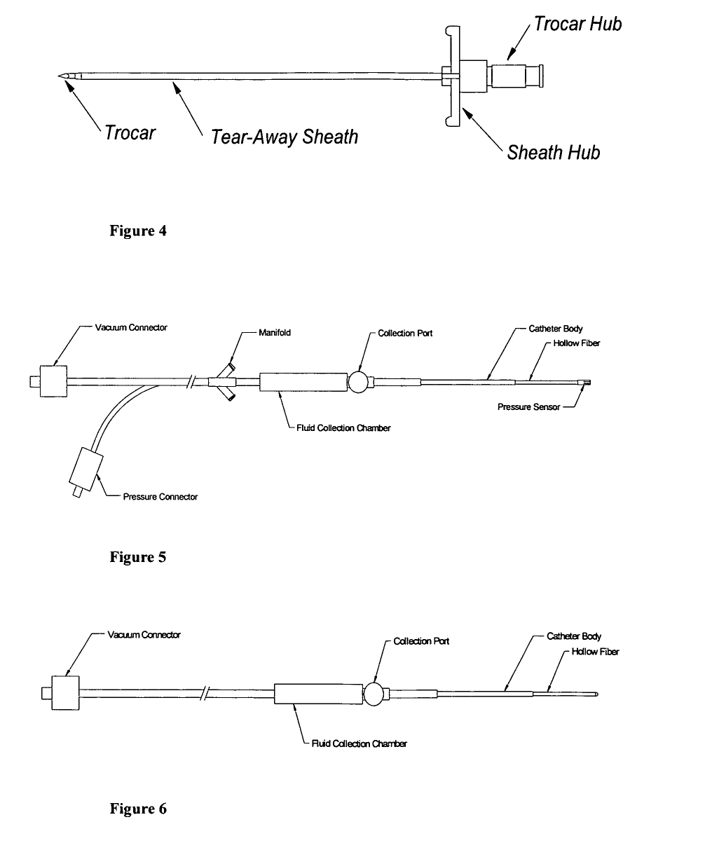

In such an embodiment, a system of this invention can include at least four components, including, an introducer, an apparatus for pressure monitoring and fluid collection, one or more fluid collection (FC) catheters, and a suitable compartment pressure monitor. Further aspects of the apparatus and corresponding system of this invention can be seen in FIGS. 4-7.

Once such introducer involves the use of a “tear-away” plastic sheath placed over a stainless steel trocar as shown in FIG. 4. The sterile disposable introducer provides access to the targeted muscle compartment to facilitate the placement of the apparatus. First, the sharp-tipped trocar and sheath are inserted through the skin and into the targeted muscle compartment. Once properly positioned, the trocar is removed leaving the hollow tear-away sheath in place. The catheter can then be placed through the hollow sheath and into the muscle compartment. Once the catheter is placed, the sheath is designed to easily tear away for removal.

The introducer's trocar and tear-away sheath design and materials can be provided in various ways that can become apparent to those skilled in the art, e.g., the tear-away sheath can be constructed of thin walled polyethylene tubing, while the trocar can be composed of stainless steel needle with a three-facet sharp tip point.

As shown, the monitoring/collection apparatus can monitor muscle compartment pressure as well as facilitate excess fluid removal, and is provided as a sterile disposable. In a particularly preferred embodiment, the apparatus includes a catheter body, hollow fiber membrane, a fiber optic pressure sensor, vacuum line and pressure sensor connectors, a catheter connection manifold, and a fluid sampling chamber with collection port as shown in FIG. 5.

The apparatus as shown provides the following functions:
Measure compartment pressure
Remove interstitial fluid
Provide fluid analysis sample
Provide connections for additional fluid collection catheters

The apparatus contains a pressure sensor at the distal tip that measures compartment pressure throughout the treatment period. The sensor can be connected to the monitor module by any suitable means, e.g., by wireless connection and signal or by an optical fiber that extends through the entire length of the catheter. A fiberoptic pressure connector is shown located at the proximal end of the apparatus, for use in connecting to a monitor.

A manifold is located just proximal to the fluid collection chamber. Two additional fluid collection (FC) catheters can be connected to the manifold using standard luer connections. An FC catheter can be designed and used to provide additional fluid collection locations within the same compartment as a monitoring/collection apparatus. The FC catheters, in turn, will typically not provide pressure measurement and are designed and intended for use with a monitoring or monitoring/recovery apparatus. The FC catheter includes a catheter body, hollow fiber membrane, vacuum line connector, and a fluid sampling chamber with collection port as shown in FIG. 6.

Interstitial fluid is removed through the hollow fiber membrane located at the distal section of the apparatus. Fluid passes through the walls of the micro-porous membrane, through the apparatus body and into the fluid collection chamber. The fluid collection chamber is connected to a vacuum line, which connects to the manifold of the apparatus. The monitor provides a low, intermittent or constant vacuum to the apparatus to draw fluid through the hollow membrane. Fluid that is contained in the collection chamber can be aspirated using a standard syringe through the collection port. The fluid can be transferred to a vial and saved for analysis.

The apparatus is designed to be used with the monitor, which can sense, display and record compartment pressure as measured by one or more such apparatuses. In addition, the monitor can be used to measure patient blood pressure using the cuff provided, which is used for calculating the perfusion pressure of the muscle compartment (Perfusion Pressure=Diastolic Blood Pressure−Compartment Pressure). The monitor can ensure the delivery of the specified functional performance needed to reliably operate the apparatus and corresponding system.

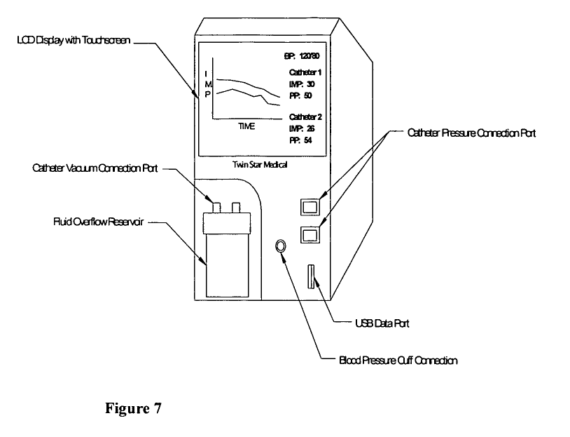

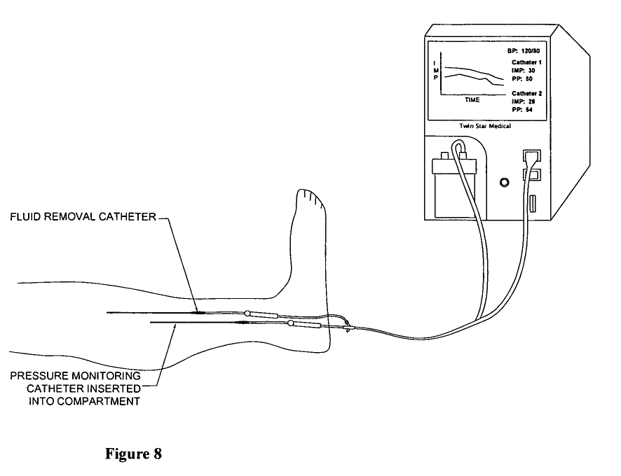

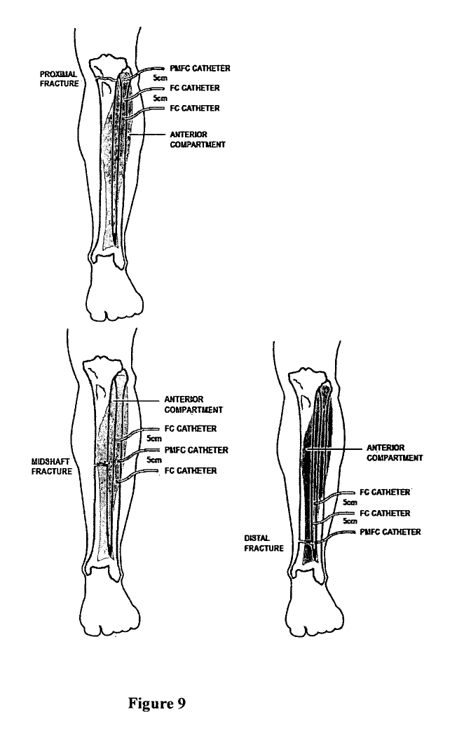

The monitor, as shown, includes a single housing that incorporates a vacuum source, a pressure monitor, a blood pressure monitor, and a user interface. An illustration of the monitor module is shown in FIG. 7. The vacuum source can draw a vacuum of up to 200 mmHg to the interstitial fluid collection line of the apparatus. The vacuum level can be set to specific values decided upon by the physician.

In addition, the module can be set to provide constant vacuum or intermittent vacuum, in order to maximize fluid removal and other parameters. The module works with pressure sensors located in the apparatus, to monitor the fluid pressure within the muscle compartment.

The module's user interface includes a touch screen display input to allow the user to add patient information, start and stop the procedure, and to save the data to a data storage device. The monitor will display the current compartment pressure and perfusion pressure for each catheter, along with a historical chart of the pressure from the start of the procedure.

A preferred pressure sensor, fluid removal system of this invention can be used in the following manner:

Surgical Procedure: The surgical procedure performed is at the discretion of the attending surgeon. Following surgery, the leg is kept elevated at the level of the heart, application of a loose compression dressing (ACE bandage), and splinting to control the position of the foot in neutral dorsiflexion.

Perioperarive Medical Care: All patients receive, as medically indicated, appropriate hydration, pain management, and other medical care as dictated by their clinical status and institutional policies.

Baseline Lab Tests: A sample (e.g., 5 cc's) is taken for baseline blood sample for future serum analyte measurements. Store serum on dry ice or in minus 70° C. freezer. Serum is then frozen for future analysis for the potential markers creatine kinase, myoglobin and potassium and can be handled and shipped per institutional guidelines.

Apparatus Insertion: The apparatus measures muscle compartment pressure by a pressure transducer located at the distal tip of the catheter. The monitor senses, displays and records pressure as measured by the catheter. In addition, the module provides vacuum required for the operation of the pressure measuring/fluid removal catheter. The catheters can be inserted at the end of the surgical procedure; the specific catheter to be inserted can be determined by the physician.

The catheters is connected to the pressure monitor and then inserted into the injured leg in the anterior compartment at the end of the surgical procedure to stabilize the tibial fracture. One apparatus can be inserted near the fracture site, while two other FC catheters can be inserted so that there is at least 5 cm of space between the catheters. In general, the catheters should be inserted from proximal to distal at an angle of 45 degrees. For tibial plateau fractures, it may be necessary to insert the apparatus from distal to proximal; the other two FC catheters may be inserted from proximal to distal. The location of the catheters can be as follows:

Proximal Third Fractures: The apparatus can be inserted first, with its tip deep in the anterior compartment muscle, within 5 cm of the primary fracture line. The two FC catheters can be inserted in the anterior compartment, one 5 cm distal to the Apparatus and one 10 cm distal to the Apparatus (see FIG. 9).

Middle Third Fractures: The apparatus can be inserted first, with its tip deep in the anterior compartment muscle, within 5 cm of the primary fracture line. The two FC catheters can be inserted in the anterior compartment, one 5 cm distal to the Apparatus and one 5 cm proximal to the Apparatus (see FIG. 9).

Distal Third Fractures: The Apparatus can be inserted first, with its tip deep in the anterior compartment muscle, within 5 cm of the primary fracture line. The two FC catheters can be inserted in the anterior compartment, one 5 cm proximal to the Apparatus and one 10 cm proximal to the Apparatus (see FIG. 9).

In a currently preferred embodiment of this invention, a physician and medical team will be able to provide an apparatus of this invention, in combination with other system components, for use in the following manner.

The catheter can be inserted as follows:

1. Turn on Monitor

2. Enter Patient ID

3. Enter patient blood pressure or attach blood pressure monitoring cuff

4. Connect the Apparatus to the monitor and “zero” the pressure transducer

5. Insert catheters into muscle compartment

6. Connect Fluid Only Catheters to the Apparatus

7. Start Acquisition

Vacuum can be applied to the catheter inner lumen for the collection of interstitial fluid. Vacuum can be continuous or discontinuous, and desired levels (e.g., continous at 150 mm Hg, or cycle 3 min. on/3 min off at 50 mm Hg).

Lab Tests: Blood samples can be taken periodically and the results used for any suitable purpose.

Patient Monitoring/Hospital Course: Patients will typically then receive standard medical care for CS monitoring i.e., muscle compartment pressure monitoring by catheter as well as standard clinical management of the condition. The Subject's length of initial hospital stay can be determined by the attending physician based on the subject's medical condition(s). Compartment syndrome is a diagnosis that depends on clinical assessment supplemented by pressure measurement. Treating physicians will monitor the patients according to standard clinical practice and will apply standard clinical judgment regarding the diagnosis of compartment syndrome.

Fasciotomy can be performed at the discretion of the attending surgeon based on his/her clinical experience and judgment in consideration of the presenting clinical signs, symptoms, and pressure measurements.

Conclusion of Monitoring Interval: After completion of the patient's treatment and/or monitoring period:
Remove the apparatus and catheters as per Instructions for Use
Discard catheters appropriately

Follow-up: A one-month and a 3-month follow-up visit can be typically be required post discharge. Functional outcomes can be measured during these follow-up as well as the occurrence of any adverse events since discharge or last follow-up. Functional outcomes will include an assessment of whether any loss of motor function resulted as part of the primary injury.

What is claimed is:

1. An apparatus comprising a functional device tip adapted to be positioned and used within a body, and in contact with a tissue site that comprises both non-fluid and fluid tissue, in a manner that permits fluid tissue to contact the tip surface but that substantially minimizes direct impingement of the non-fluid tissue on the tip surface.

2. The apparatus of claim 1, wherein the functional tip comprises a sensor having a sensor surface, the sensor being positioned within a catheter, for use in determining one or more tissue parameters, the catheter being configured and used in a manner that permits the apparatus to be positioned in tissue in a manner that permits the sensor to effectively contact fluid within the site, but that minimizes the extent to which the sensor can be impinged upon by non-fluid tissue itself.

3. The apparatus of claim 2, wherein the catheter further comprises one or more conduits which provide the ability to either deliver and/or remove fluids and/or components thereof, for therapeutic and/or analytical purposes.

4. The apparatus of claim 3 wherein the conduits comprise one or more hollow semipermeable membranes.

5. The apparatus of claim 3 wherein the timing and position of delivery and/or removal can be adjusted in response to the use of the functional tip.

6. The apparatus of claim 1, wherein the apparatus is adapted and use solely for the purpose of sensor placement and use.

7. The apparatus of claim 1 wherein the sensor is selected from the group consisting of sensor for use in monitoring pressure, pH, temperature, oxygenation, potassium or other electrolytes, biomarkers, optical spectroscopy parameters, and tissue impedance.

8. The apparatus of claim 7 wherein the sensor is a pressure sensor operates based on a technology selected from the group consisting of fiber optics, electronic chips, and ultrasound.

9. The apparatus of claim 1 wherein the functional device tip provides a function selected from the group consisting of heat, ultrasound and/or an electrical signal sufficient to treat the corresponding tissue site or surface.

10. The apparatus of claim 2 wherein the sensor provides an optimal combination of such features as device tip durability and reliability, ease of use, miniature size, mounting flexibility, resolution, consistency, accuracy and precision, reading rate, fast response, and immunity to electromagnetic field or radiofrequency interference.

11. The apparatus of claim 1 wherein the apparatus further comprises one or more lumen for removing fluid from the tissue site.

12. The apparatus of claim 1 wherein the lumen comprise one or more semi permeable membranes in order to permit sampling and/or removal of fluid from the tissue site.

13. A system comprising the apparatus of claim 1 in combination with one or more components selected from the group selected from monitors for monitoring the function of the functional device tip, means for determining and/or controlling the placement or use of the apparatus, and pumps, conduits and containers for delivering and/or removing fluids or other materials to or from the tissue site by means of the apparatus.

14. A method of using a functional device tip to be positioned and used within a body, and in contact with a tissue site that comprises both non-fluid and fluid tissue, in a manner that permits fluid tissue to contact the tip surface but that substantially minimizes direct impingement of the non-fluid tissue on the tip surface, the method comprising the step of fabricating an apparatus according to claim 1 and inserting the apparatus into the tissue site.
